(19) United States
(12) Patent Application Publication
(10) Pub. No.: US 2025/0344968 A1
(43) Pub. Date: Nov. 13, 2025

(54) Clinical Bedside Systems and Methods with Biosensors for Complex Care Patients

(71) Applicant: Bard Access Systems, Inc., Salt Lake City, UT (US)

(72) Inventors: James L. Freasier, Salt Lake City, UT (US); Ejaz S. Haque, Salt Lake City, UT (US); Jorge Jimenez, Salt Lake City, UT (US); Brendan Laine, Salt Lake City, UT (US); Megan Wimmer, Ellicott City, MD (US)

(21) Appl. No.: 19/205,859

(22) Filed: May 12, 2025

Related U.S. Application Data
(60) Provisional application No. 63/646,291, filed on May 13, 2024.

(51) Int. Cl. A61B 5/145; A61B 5/00; A61B 5/1486

(52) U.S. Cl. CPC A61B 5/14546; A61B 5/14865; A61B 5/6852; A61B 2560/0468

(57) ABSTRACT

A clinical bedside system facilitates care for complex care patients. The system can include a medical device having a biosensor with an inert substrate and a working electrode, a counter electrode, and a reference electrode deposited thereon. The working electrode can have an antifouling membrane thereover to which a capture antibody is immobilized. The capture antibody can be configured to capture a biomarker between it and a detection antibody, thereby sandwiching the infection biomarker between the antibodies for a detectable redox reaction between an enzyme conjugated to the detection antibody and the working electrode. The counter electrode can complete completes an electrical circuit including the working electrode. The reference electrode can be operably connected to the electrical circuit. The reference electrode can be configured to provide a reference point against which changes in potential at the working electrode can be measured, for example, with a potentiostat.

### PRIORITY

This application claims the benefit of priority to U.S. Provisional Application No. 63/646,291, filed May 13, 2024, which is incorporated by reference in its entirety into this application.

### BACKGROUND

Reliable biomarkers for the diagnosis and prognosis of infections including sepsis are critical. Ideal biomarkers should be highly specific for one or more infections, detectable at low concentrations, and easy to determine analytically. Using such biomarkers would not only provide early diagnostic accuracy and prognostic information on infections but also predict the responsiveness to treatment interventions.

The interleukin (“IL”)-6, IL-10, and procalcitonin (“PCT”) tests all have a high diagnostic value for patients with sepsis, and the combination of the foregoing tests outperforms any individual tests in terms of diagnostic performance and overall clinical benefit rate. Additionally, studies and clinical meta-analysis of biomarkers have confirmed that IL-6, IL-10, and PCT are efficient biomarkers for distinguishing between blood-stream infections (“BSI”) and other types of infections. Furthermore, they can be utilized to classify BSI pathogens and differentiate between vancomycin-resistant enterococcus (“VRE”) and vancomycin-susceptible enterococcus (“VSE”). The ability to provide clinical bedside monitoring of these biomarkers through ‘first-to-access’ devices can substantially improve the standard of care for treating infections including sepsis.

Disclosed herein are clinical bedside systems and methods that address the foregoing.

### SUMMARY

Disclosed herein is a clinical bedside system for complex care patients. The system includes, in some embodiments, a medical device including a biosensor. The biosensor includes an inert substrate, optionally, including a portion of the medical device itself; one or more working electrodes (“working electrode[s]”); a counter electrode; and, optionally, a reference electrode. The working electrode(s) are deposited on the substrate. Each working electrode of the working electrode(s) has an antifouling membrane thereover to which a capture antibody or a homogenous population of capture antibodies including the capture antibody is immobilized. The capture antibody is configured to capture a biomarker between it and a detection antibody of a homogenous population of detection antibodies including the detection antibody. Capture of the biomarker between the capture antibody and the detection antibody thusly sandwiches the biomarker between the capture antibody and the detection antibody for a detectable redox reaction between an enzyme conjugated to the detection antibody and its corresponding working electrode. The counter electrode is deposited on the substrate such that the counter electrode completes one or more electrical circuits (“electrical circuit[s]”) including the working electrode(s), respectively. When present, the reference electrode is deposited on the substrate such that the reference electrode is operably connected to the electrical circuit(s). The reference electrode is configured to provide a reference point against which changes in potential at the working electrode(s) are measured.

In some embodiments, the system further includes a potentiostat configured to modulate the potential at any working electrode of the working electrode(s) and measure current in its corresponding electrical circuit. A magnitude of the current is proportional to a concentration of the biomarker in a biological fluid to which the biosensor or its working electrode(s) are exposed.

In some embodiments, cach electrode of the working electrode(s), the counter electrode, and the reference electrode is independently formed of gold or platinum.

In some embodiments, the antifouling membrane over each working electrode of the working electrode(s) includes a conducting polymer independently selected from polyethylenimine; poly(3-methylthiophene); poly-5,2′-5′,2″-terthiophene-3′-carboxylic acid; polyaniline; polyaniline-poly(acrylic acid); polypyrrole-polyvinyl sulfonate; polyanion-doped poly(pyrrole); and poly(o-phenylenediamine).

In some embodiments, the capture antibody is either directly immobilized on the antifouling membrane or indirectly immobilized on the antifouling membrane through streptavidin or avidin.

In some embodiments, the capture antibody is a biotinylated antibody.

In some embodiments, the enzyme conjugated to the detection antibody is horseradish peroxidase (“HRP”), alkaline phosphatase (“ALP”), β-galactosidase, acetylcholinesterase (“AchE”), or catalase.

In some embodiments, each antibody of the capture antibody and the detection antibody is an anti-biomarker antibody for recognizing the biomarker and sandwiching the biomarker between the capture antibody and the detection antibody.

In some embodiments, the biomarker is a pro-inflammatory mediator selected from an interleukin including at least IL-1α, IL-1B, IL-6, IL-8, IL-11, IL-12, IL-17, IL-18, a member of the IL-20 family, or IL-33; tumor necrosis factor-α (“TNF-α”); leukemia inhibitory factor (“LIF”); interferon-γ (“IFN-γ”); oncostatin M (“OSM”); ciliary neurotrophic factor (“CNTF”); transforming growth factor-β (“TGF-β”); granulocyte macrophage colony-stimulating factor (“GM-CSF”); and other immunoregulatory biomolecules that attract inflammatory cells.

In some embodiments, the biomarker is an anti-inflammatory mediator selected from an interleukin including at least IL-1 receptor antagonist (“IL-1Ra”), IL-4, IL-6, IL-10, IL-11, or IL-13; and other immunoregulatory biomolecules that prevent potentially harmful effects of persistent or excess inflammatory reactions.

In some embodiments, the biomarker is PCT, lactate, or C-reactive protein (“CRP”).

In some embodiments, the biomarker is angiopoietin-1 (“ANG-1”), angiopoietin-2 (“ANG-2”), plasminogen activator inhibitor-1 (“PAI-1”), tissue inhibitor of metalloproteinase-2 (“TIMP-2”), insulin-like growth factor-binding protein-7 (“IGFBP7”), soluble tumor necrosis factor receptor-1 (“sTNFR1”), receptor for advanced glycation endproducts (“RAGE”), decoy receptor 3 (“Dcr3”), soluble CD163, delta-like protein 1 (“DLL1”), hyaluronan, or syndecan.

In some embodiments, each working electrode of the working electrode(s) is configured to detect a unique biomarker, thereby providing a suite of biomarkers for both precision and accuracy.

In some embodiments, the medical device is a central venous catheter, an indwelling urinary catheter, or a skin-contacting wearable medical device.

Also disclosed herein is a biosensor for complex care patients. The biosensor includes, in some embodiments, an inert substrate; one or more working electrodes deposited on the substrate; a counter electrode; and an optional reference electrode. The working electrode(s) are deposited on the substrate. Each working electrode of the working electrode(s) has an antifouling membrane thereover to which a capture antibody or a homogenous population of capture antibodies including the capture antibody is immobilized. The capture antibody is configured to capture a biomarker between it and a detection antibody of a homogenous population of detection antibodies including the detection antibody. Capture of the biomarker between the capture antibody and the detection antibody thusly sandwiches the biomarker between the capture antibody and the detection antibody for a detectable redox reaction between an enzyme conjugated to the detection antibody and its corresponding working electrode. The counter electrode is deposited on the substrate such that the counter electrode completes one or more electrical circuits including the working electrode(s), respectively. When present, the reference electrode is deposited on the substrate such that the reference electrode is operably connected to the electrical circuit(s). The reference electrode is configured to provide a reference point against which changes in potential at the working electrode(s) are measured.

In some embodiments, cach electrode of the working electrode(s), the counter electrode, and the reference electrode is independently formed of gold or platinum.

In some embodiments, the antifouling membrane over each working electrode of the working electrode(s) is a conducting polymer independently selected from polyethylenimine; poly(3-methylthiophene); poly-5,2′-5′,2″-terthiophene-3′-carboxylic acid; polyaniline; polyaniline-poly(acrylic acid); polypyrrole-polyvinyl sulfonate; polyanion-doped poly(pyrrole); and poly(o-phenylenediamine).

In some embodiments, the capture antibody is either directly immobilized on the antifouling membrane or indirectly immobilized on the antifouling membrane through streptavidin or avidin.

In some embodiments, the capture antibody is a biotinylated antibody.

In some embodiments, the enzyme conjugated to the detection antibody is HRP, ALP, β-galactosidase, AchE, or catalase.

In some embodiments, each antibody of the capture antibody and the detection antibody is an anti-biomarker antibody for recognizing the biomarker and sandwiching the biomarker between the capture antibody and the detection antibody.

In some embodiments, the biomarker is a pro-inflammatory mediator selected from an interleukin including at least IL-1α, IL-1β, IL-6, IL-8, IL-11, IL-12, IL-17, IL-18, a member of the IL-20 family, or IL-33; TNF-α; LIF; IFN-γ; OSM; CNTF; TGF-β; GM-CSF; and other immunoregulatory biomolecules that attract inflammatory cells.

In some embodiments, the biomarker is an anti-inflammatory mediator selected from an interleukin including at least IL-1Ra, IL-4, IL-6, IL-10, IL-11, or IL-13; and other immunoregulatory biomolecules that prevent potentially harmful effects of persistent or excess inflammatory reactions.

In some embodiments, the biomarker is procalcitonin PCT, lactate, or CRP.

In some embodiments, each working electrode of the working electrode(s) is configured to detect a unique biomarker, thereby providing a suite of biomarkers for both precision and accuracy.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a schematic of a clinical bedside system including a biosensor and a potentiostat in accordance with some embodiments.

FIG. 2 illustrates a schematic of a working electrode of the biosensor in accordance with some embodiments.

FIG. 3 illustrates an example of a cyclic voltammogram for a detectable redox reaction at the working electrode in accordance with some embodiments.

FIG. 4 illustrates a catheter including the biosensor in accordance with some embodiments.

FIG. 5 illustrates a detailed view of the biosensor in the catheter in accordance with some embodiments.

FIG. 6 illustrates a wearable patch including the biosensor in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Complex care patients,” as used herein, includes patients having one or more, often multiple, medical conditions requiring an integrated approach to care spanning primary and specialty services. By way of example, such medical conditions include, but are not limited to, acute respiratory distress syndrome (“ARDS”), sepsis, ventilated pneumonia, medical conditions requiring arterial catheters, acute kidney injury (“AKI”), and trauma.

Unless defined otherwise, all technical and scientific terms used herein have the same meaning as commonly understood by those of ordinary skill in the art.

### Systems

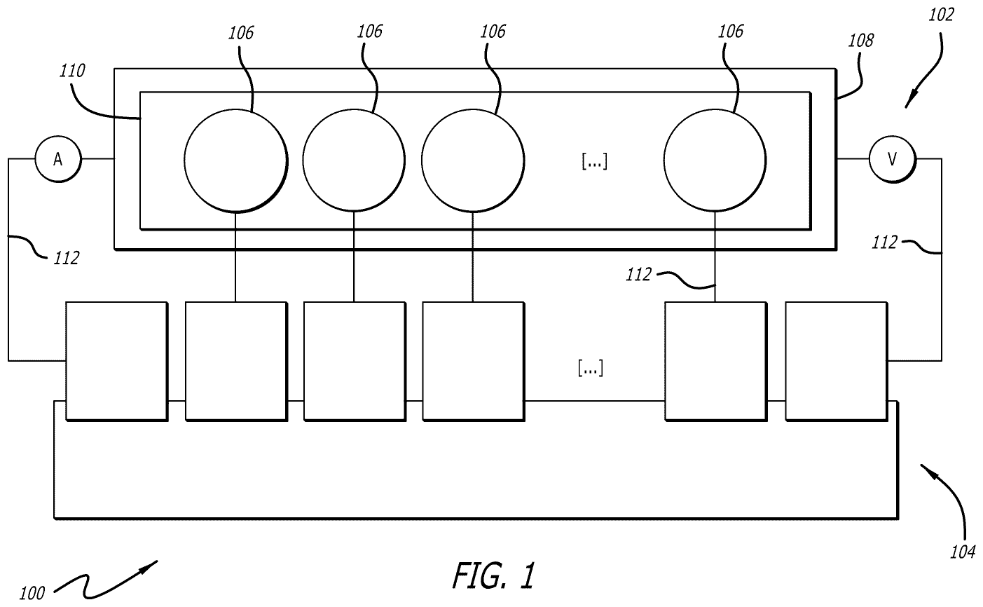

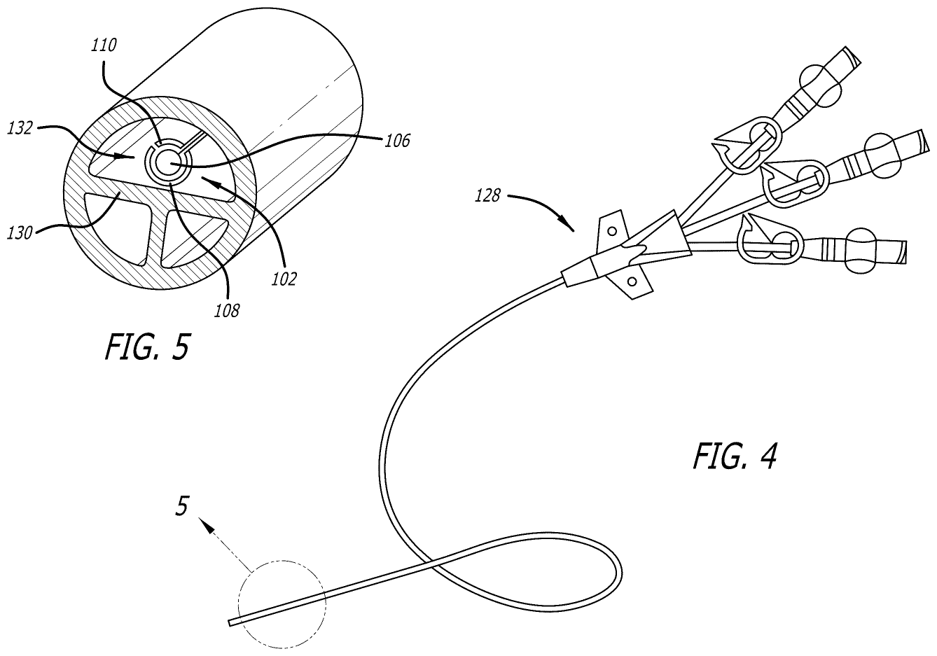

FIG. 1 illustrates a schematic of a clinical bedside system 100 including at least one biosensor 102 and a potentiostat 104 in accordance with some embodiments. FIGS. 4-6 illustrate example medical devices of the system 100 including the biosensor 102 in accordance with some embodiments.

As shown, the system 100 for complex care patients can include a medical device having the biosensor 102 or a plurality of such biosensors and, optionally, the potentiostat 104. While not shown, the system 100 can include a console including the potentiostat 104 or operably connected to the potentiostat 104 for automatically determining concentrations of biomarkers 114 in biological fluids and whether the concentrations are indicative of infection. Thus, the system 100 can be defined in terms of a single-use medical device having at least the biosensor 102 or the single-use medical device and some capital equipment such as the potentiostat 104, the console, or some combination thereof.

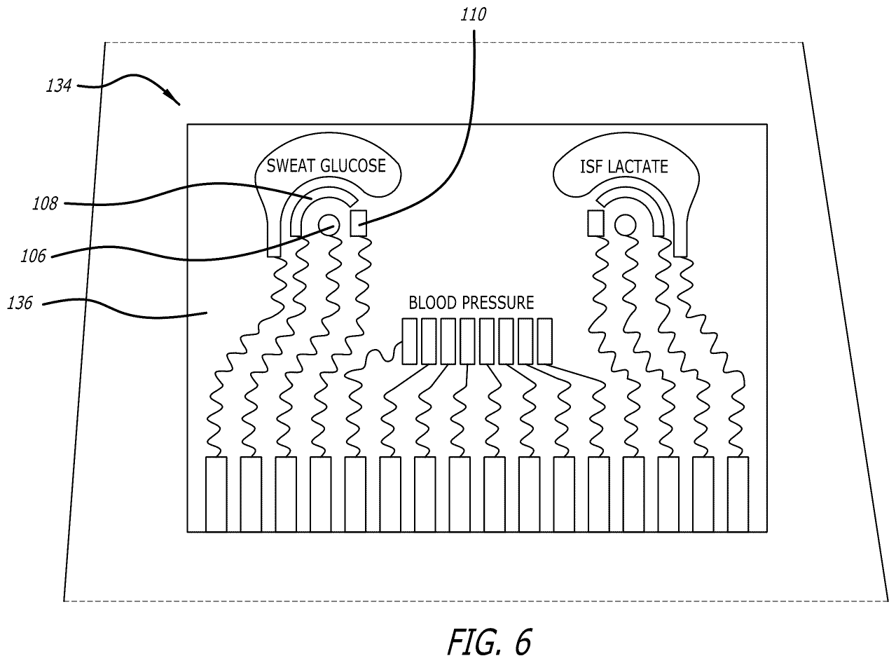

Beginning with the biosensor 102, the biosensor 102 can include an inert substrate such as a portion of the medical device itself, a rigid glass substrate, a flexible polymer substrate, etc. Indeed, as shown in FIG. 5, the biosensor 102 can be disposed on the septum 130 within the primary or distal lumen 132 of the catheter 128, the septum 130 being the foregoing portion of the medical device itself. Further, as shown in FIG. 6, the biosensor 102 can be disposed on the adherable skin-facing side of the covering 136 of the wearable patch 134, the covering 136 being the foregoing flexible polymer substrate.

The biosensor 102 can also include one or more working electrodes 106, a counter electrode 108, and, optionally, a reference electrode 110 deposited on the substrate. In an example, the biosensor 102 can include as few as two electrodes when the biosensor 102 includes a single working electrode 106 and the counter electrode 108. In another example, the biosensor 102 can include three electrodes when the biosensor 102 includes two working electrodes 106 and the counter electrode 108 or the single working electrode 106, the counter electrode 108, and the reference electrode 110. In another example, the biosensor 102 can include more than three electrodes when the biosensor 102 includes three working electrodes 106 and the counter electrode 108 or at least two working electrodes 106, the counter electrode 108, and the reference electrode 110. Notably, the counter electrode 108 is deposited on the substrate such that the counter electrode 108 completes one or more electrical circuits 112 including the working electrode(s) 106, respectively. When present, the reference electrode 110 is deposited on the substrate such that the reference electrode 110 is operably connected to the electrical circuit(s) 112, which reference electrode 110 advantageously provides a reference point against which changes in potential at the working electrode(s) 106 can be detected or measured.

Each working electrode 106 of the working electrode(s) 106 in the biosensor 102 can be configured for detecting or measuring a same or different biomarker 114 than another working electrode 106 of the working electrode(s) 106. When any two or more working electrodes 106 are configured to detect or measure the same biomarker 114, there is redundancy in detecting or measuring the foregoing biomarker 114. When any two or more working electrodes 106 are configured to detect or measure different or unique biomarkers 114, there is multiplexity in detecting or measuring the different biomarkers 114 for differentiating between various infections. Indeed, a plurality of working electrodes 106 can be configured to detect or measure any combination of the biomarkers 114 set forth below, with or without redundancy, for a multiplex biosensor 102 for differentiating between various infections. Such a multiplex biosensor 102 can thusly provide a suite of biomarkers 114 for increased performance in both precision and accuracy, which can be useful in the early detection of a particular infection such as sepsis.

Each electrode of the working electrode(s) 106, the counter electrode 108, and the reference electrode 110 can have a conductive element 116 independently formed of a metal such as titanium, nickel, copper, zinc, silver, platinum, or gold or an alloy such as brass. However, at least the working electrode(s) 106 can alternatively be an indium-tin-oxide (“ITO”)-coated; a carbon electrode such as a glassy carbon electrode (“GCE”) or a polymer-modified GCE (e.g., a poly[3-methylthiophene]-modified GCE); a polysulfone screen-printed electrode; a gold coated SiO₂ electrode; or a poly(acrylic acid)/Si₃N₄ composite or nanostructured electrode, the conductive element 116 of such a working electrode 106 being suitably configured therefor.

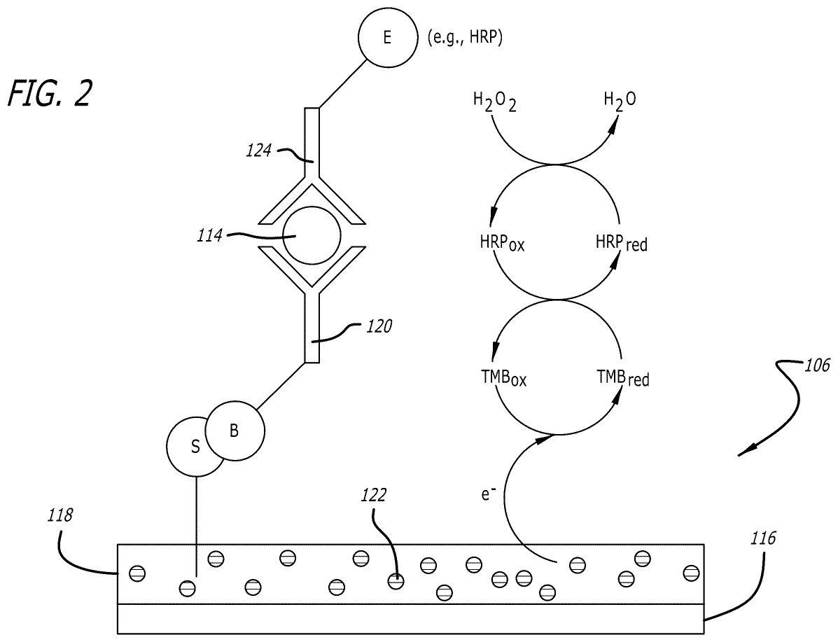

FIG. 2 illustrates a schematic of an example working electrode 106 of the biosensor 102.

Each working electrode 106 of the working electrode(s) 106 can have an antifouling membrane 118 thereover to which a unique or duplicative capture antibody 120 or a homogenous population of capture antibodies 120 including the foregoing capture antibody 120 is immobilized. The antifouling membrane 118 over each working electrode 106 of the working electrode(s) 106 can be a biomimetic membrane (e.g., denatured proteins, hydrogel, etc.) modified with the electronics-modifying agent 122 set forth below, a conducting-polymer membrane, or some combination thereof such as a composite membrane. A conducting polymer for the biomimetic membrane can be independently selected from polyethylenimine; poly(3-methylthiophene); poly-5,2′-5′,2″-terthiophene-3′-carboxylic acid; chitosan (when wetted during use); polyaniline; polyaniline-poly(acrylic acid); polypyrrole-polyvinyl sulfonate; polyanion-doped poly(pyrrole); poly(vinyl imidazole); and poly(o-phenylenediamine). The antifouling membrane 118 over each working electrode 106 of the working electrode(s) 106 can alternatively or additionally independently include photocatalytically reduced graphene (“PRG”), carbon nanotubes such as single walled carbon nanotubes (“SWCNTs”), multi-walled carbon nanotubes (“MWCNTs”), or hydrogen titanate nanotubes (“HTNTs”). When the antifouling membrane 118 additionally includes such PRG, SWCNTs, MWCNTs, or HTNTs, the PRG, SWCNTs, MWCNTs, or HTNTs can be one or more additional layers of the antifouling membrane 118, form a composite membrane with any of the foregoing conducting polymers, or some combination thereof. The antifouling membrane 118 over cach working electrode 106 of the working electrode(s) 106 can independently include an electronics-modifying agent 122 for modifying the electronics of the antifouling membrane 118 such as an electron-transfer-facilitating agent. Such an electronics-modifying agent 122 can include, but is not limited to, a nitrogen-based dopant; ferrocyanide; ferrocene; nanoparticles of one or more metals including gold nanoparticles such as gold nanorods or platinum nanoparticles such those of platinum black; nanoparticles of one or more metal oxides including titanium dioxide nanoparticles, iron oxide nanoparticles, zinc oxide nanoparticle such as zinc oxide nanorods, nanowires, or nanotetrapods, niobium oxide nanoparticles, molybdenum oxide nanoparticles such as molybdenum oxide nanowires, or cerium oxide nanoparticles; or Meldola's Blue integrated in or adsorbed on the antifouling membrane 118.

Again, each working electrode 106 of the working electrode(s) 106 can have an antifouling membrane 118 thereover to which a unique or duplicative capture antibody 120 or a homogenous population of capture antibodies 120 including the foregoing capture antibody 120 is immobilized. The capture antibody 120 can be directly immobilized on the antifouling membrane 118 via chemical coupling or physical adsorption, or the capture antibody 120 can be indirectly immobilized on the antifouling membrane 118 through, for example, a biotin B interaction with streptavidin S, avidin, or the like when the capture antibody 120 is biotinylated as shown in FIG. 2. The capture antibody 120 can be configured to capture a biomarker 114 between it and a corresponding free (i.e., not captured) detection antibody 124.

Like that set forth above for the capture antibody 120, the detection antibody 124 can be part of a homogenous population of detection antibodies including the foregoing detection antibody 124, which can be continuously introduced to the biosensor 102 for continuous measurement or periodically introduced to the biosensor 102 for periodic measurements by way of flushing the biosensor 102 with a detection-antibody solution. Capture of a biomarker 114 between a capture antibody 120 and its corresponding detection antibody 124 sandwiches the biomarker 114 between the capture antibody 120 and the detection antibody 124, which, in turn, allows for a detectable redox reaction between a redox reactant such as an enzyme E conjugated to the detection antibody 124 and the working electrode 106 including the capture antibody 120 when the potential of the working electrode 106 is a swept in accordance with cyclic voltammetry.

Each antibody of the capture antibody 120 and the detection antibody 124 in a pair of corresponding antibodies can be an anti-biomarker antibody for recognizing a corresponding biomarker 114 and sandwiching the biomarker 114 between the capture antibody 120 and the detection antibody 124. It should be understood that “anti,” as in “anti-biomarker antibody,” is a prefix indicating any biomarker that follows “anti” is the biomarker 114 for which the capture antibody 120 and the detection antibody 124 in a pair of corresponding antibodies is configured to recognize. In an example, the biomarker 114 can be procalcitonin (“PCT”), as set forth below, and each antibody of the capture antibody 120 and the detection antibody 124 in a pair of corresponding antibodies can be an anti-PCT antibody configured to recognize PCT.

The biomarker 114 that the capture antibody 120 and the detection antibody 124 in a pair of corresponding antibodies are configured to recognize can include a pro-inflammatory mediator selected from at least an interleukin (“IL”) including at least IL-1α, IL-1β, IL-6, IL-8, IL-11, IL-12, IL-17, IL-18, a member of the IL-20 family, or IL-33; tumor necrosis factor-α (“TNF-α”); leukemia inhibitory factor (“LIF”); interferon-γ (“IFN-γ”); oncostatin M (“OSM”); ciliary neurotrophic factor (CNTF); transforming growth factor-β (“TGF-β”); granulocyte macrophage colony-stimulating factor (“GM-CSF”); and other immunoregulatory biomolecules that attract inflammatory cells.

The biomarker 114 that the capture antibody 120 and the detection antibody 124 in a pair of corresponding antibodies are configured to recognize can include an anti-inflammatory mediator selected from at least an interleukin including at least IL-1 receptor antagonist (“IL-1Ra”), IL-4, IL-6, IL-10, IL-11, or IL-13; and other immunoregulatory biomolecules that prevent potentially harmful effects of persistent or excess inflammatory reactions.

The biomarker 114 that the capture antibody 120 and the detection antibody 124 in a pair of corresponding antibodies are configured to recognize can include at least PCT, lactate, or C-reactive protein (“CRP”).

The biomarker 114 that the capture antibody 120 and the detection antibody 124 in a pair of corresponding antibodies are configured to recognize can include angiopoietin-1 (“ANG-1”), angiopoietin-2 (“ANG-2”), plasminogen activator inhibitor-1 (“PAI-1”), tissue inhibitor of metalloproteinase-2 (“TIMP-2”), insulin-like growth factor-binding protein-7 (“IGFBP7”), soluble tumor necrosis factor receptor-1 (“sTNFR1”), receptor for advanced glycation endproducts (“RAGE”), decoy receptor 3 (“Dcr3”), soluble CD163, delta-like protein 1 (“DLL1”), hyaluronan, or syndecan.

Notably, it has been documented that IL-6 production is elevated in patients with sepsis, thereby indicating that IL-6 is associated with the development of sepsis. Further studies have indicated that the IL-6 level is higher in patients with shock than those without shock and in those who died from severe sepsis, thereby suggesting that IL-6 is a key cytokine in the pathophysiology of severe sepsis. In addition, an increased level of IL-6 was found to be associated with the highest risk of death in patients with sepsis. Among the many cytokines induced during sepsis, plasma IL-6 has one of the strongest correlations with mortality rate.

Further, it has been reported that IL-10 is a notable cytokine in the pathophysiology of sepsis. Indeed, measurement of serum cytokines in patients with severe sepsis indicated that the IL-10 level was significantly elevated. Increased IL-10 levels in serum correlated with the sepsis score and death. A high IL-10-to-TNF-α ratio was associated with death. In addition, persistent overproduction of IL-10 is a main risk factor for sepsis severity and fatal outcome, suggesting that patients with sepsis are in profound immunosuppression.

Further, PCT is a peptide precursor of calcitonin, which participates in calcium homeostasis. It has been demonstrated that serum levels of PCT rise dramatically in response to bacterial infection. Thus, measurement of PCT can be used as a marker of severe sepsis such as that caused by bacteria. PCT is also detectable at low concentrations, and PCT is specific for differentiating patients with Systemic Inflammatory Response Syndrome (“SIRS”) from those with sepsis when compared with IL-2, IL-6, IL-8 and TNF-α. Currently, PCT assays are used in clinical settings but there are limitations in that PCT signals in such assays need amplification, which takes time and costly kits and reagents.

Again, capture of a biomarker 114 between a capture antibody 120 and its corresponding detection antibody 124 sandwiches the biomarker 114 between the capture antibody 120 and the detection antibody 124, which allows for a detectable redox reaction between a redox reactant such as the enzyme E conjugated to the detection antibody 124 and the working electrode 106 including the capture antibody 120 when the potential of the working electrode 106 is a swept in accordance with cyclic voltammetry. As shown in FIG. 2, for example, such an enzyme can be horseradish peroxidase (“HRP”); however, the enzyme can alternatively be alkaline phosphatase (“ALP”), β-galactosidase, acetylcholinesterase (“AchE”), catalase, or some other enzyme configure to redox couple with a working electrode 106 when the potential of the working electrode 106 is a swept in accordance with cyclic voltammetry. Advantageously, HRP can redox couple to a chromogenic species such as 3,3′,5,5′-tetramethylbenzidine (“TMB”) per the illustrated redox cycle to provide a chromogenic response through a charge-transfer complex of TMB and an oxidized form thereof. Alternatively, Variamine blue can be used to provide a chromogenic response with HRP or another enzyme to which it can form a redox couple. Notably, while a chromogenic response provides an instant visual indication of a biomarker 114, the chromogenic response can also advantageously be colorimetrically analyzed with a colorimeter to complement any cyclic voltammogram from the potentiostat 104.

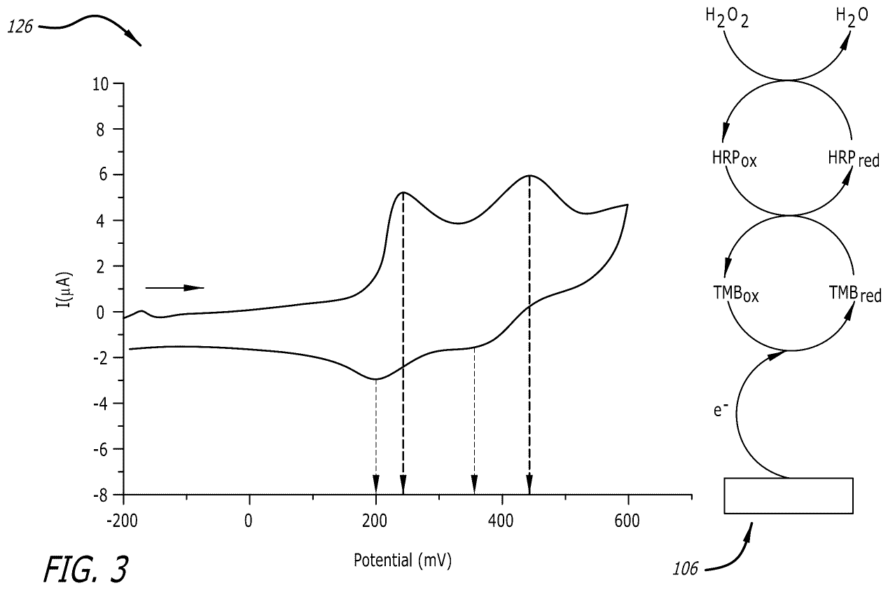

FIG. 3 illustrates an example of a cyclic voltammogram 126 for a detectable redox reaction at the working electrode 106 in accordance with some embodiments.

As set forth above, the system 100 for complex care patients can include the potentiostat 104, which is configured to modulate the potential at any working electrode 106 of the working electrode(s) 106 and measure current in its corresponding electrical circuit 112. Measurement data (i.e., current vs. potential) for any working electrode 106 of the working electrode(s) 106 can be plotted as shown in FIG. 3 to generate a cyclic voltammogram. Not only can a cyclic voltammogram represent a fingerprint for each working electrode 106 of the working electrode(s) 106 corresponding to its particular redox-coupling design, but a magnitude of the current such as a magnitude of a peak current in a cyclic voltammogram is proportional to a concentration of the biomarker 114 in a biological fluid such as blood, urine, saliva, sweat, or tears to which the biosensor 102 or its working electrode(s) 106 are exposed. Indeed, the concentration of the biomarker 114 in the biological fluid can be determined by checking the magnitude of the peak current against a plot or calibration curve of current vs. concentration of the biomarker 114, which can be accomplished manually or automatically through the console including the potentiostat 104 or operably connected to the potentiostat 104. If the concentration of the biomarker 114 or several biomarkers 114 are elevated against threshold values therefor for an infection, the infection is indicated.

Notwithstanding the foregoing, it should be understood that the biosensor 102 set forth above can be modified to additionally or alternatively include optical immunosensors, piezoelectric immunosensors, biosensor field-effect transistors (“Bio-FETs”), or some combination thereof, each of which can utilize at least one biological recognition element of the capture antibody 120 or the detection antibody 124 set forth above.

Again, FIGS. 4-6 illustrate example medical devices of the system 100 including the biosensor 102 in accordance with some embodiments.

As shown in FIGS. 4 and 5, the medical device can be a catheter 128 including the biosensor 102. Such a catheter 128 can include a venous catheter such as a peripheral venous catheter or a central venous catheter (“CVC”); a urinary catheter such as an intermittent catheter or an indwelling urinary catheter; or the like. By way of example, a multiluminal CVC is shown in FIGS. 4 and 5 for the foregoing venous catheter, wherein the biosensor 102 is disposed on a septum 130 within a primary or distal lumen 132 of the CVC where the working electrode(s) 106 can interact with blood when blood is drawn through the CVC. If the catheter 128 is a urinary catheter instead of such a venous catheter, the biosensor 102 can likewise be disposed within a drainage lumen of the urinary catheter where the working electrode(s) 106 can interact with urine when urine is drained from the urinary catheter.

Relatedly, the medical device can alternatively be a sampling or collection device for blood or urine. For example, the medical device can include the PIVO™ Pro Needle-free Blood Collection Device (BD, Franklin Lakes, NJ). Akin to the foregoing venous catheter, the biosensor 102 can be disposed within the lumen of the flow tube of the PIVO™ Pro Needle-free Blood Collection Device where the working electrode(s) 106 can interact with blood when blood is drawn through therethrough.

Further, any medical device of the foregoing medical devices, whether the medical device is a venous catheter, a urinary catheter, or a sampling or collection device for blood or urine, can include an optical-fiber stylet or the like disposed in the medical device for transmitting a chromogenic response of the biosensor 102 within the medical device to an observer or detector outside the medical device. In an example, the optical-fiber stylet can extend from the biosensor 102 within the medical device to a detection window on an outside of the medical device or observation by the observer. In another example, the optical-fiber stylet can extend from the biosensor 102 within the medical device through an extension tube of the medical device for connection to the detector for amplifying the chromogenic response, colorimetrically analyzing the chromogenic response, or both.

As shown in FIG. 6, the medical device can be a wearable patch 134 including the biosensor 102. Such a wearable patch 134 can include the biosensor 102 disposed on an adherable skin-facing side of a covering 136 of the wearable patch 134 where the working electrode(s) 106 can interact with sweat when sweat is generated by a wearer of the wearable patch 134.

Relatedly, the medical device can alternatively be a securement device such as the Statlock® PICC/CVC Stabilization Device (BD, Franklin Lakes, NJ) or a wound dressing, itself, or in operable combination with the securement device.

Lastly, the medical device can be a mouthguard including the biosensor 102. Such a mouthguard can include the biosensor 102 disposed on a free surface of the mouthguard (e.g., a surface that is not intended to contact teeth) where the working electrode(s) 106 can interact with saliva generated by a wearer of the mouthguard.

Notwithstanding the foregoing, it should be understood that the biosensor 102 can, in some embodiments of the system 100, instead be incorporated into a test kit rather than the medical device. Such a test kit can be configured to test any biological fluid selected from blood, urine, saliva, sweat, and tears to which the biosensor 102 or its working electrode(s) 106 are exposed.

### Methods

Methods include methods of making and using the systems and methods set forth herein, which can be gleaned from description of various structural components and their arrangements with respect to each other as well as their stated functions.

Advantageously, the systems and methods set forth herein solve issues with current practices by enabling bedside analysis of biological fluids to infer infections such as sepsis, and, in the case of sepsis, determine whether it is based in a microbial infection. Solving such issues can streamline decision making for clinicians so they can treat microbial-induced sepsis with antibiotics or pursue treatment of non-infection related sepsis through other means.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A clinical bedside system for complex care patients, comprising:
a medical device including:
a biosensor including:
an inert substrate, optionally, including a portion of the medical device itself;
one or more working electrodes (“working electrode[s]”) deposited on the substrate, each working electrode of the working electrode(s) having an antifouling membrane thereover to which a capture antibody or a homogenous population of capture antibodies including the capture antibody is immobilized, the capture antibody configured to capture a biomarker between it and a detection antibody of a homogenous population of detection antibodies including the detection antibody, thereby sandwiching the biomarker between the capture antibody and the detection antibody for a detectable redox reaction between an enzyme conjugated to the detection antibody and its corresponding working electrode;
a counter electrode deposited on the substrate such that the counter electrode completes one or more electrical circuits (“electrical circuit[s]”) including the working electrode(s), respectively; and
an optional reference electrode deposited on the substrate such that the reference electrode is operably connected to the electrical circuit(s), the reference electrode configured to provide a reference point against which changes in potential at the working electrode(s) are measured.

2. The clinical bedside system of claim 1, further comprising a potentiostat configured to modulate the potential at any working electrode of the working electrode(s) and measure current in its corresponding electrical circuit, a magnitude of the current proportional to a concentration of the biomarker in a biological fluid to which the biosensor or its working electrode(s) are exposed.

3. The clinical bedside system of claim 1, wherein each electrode of the working electrode(s), the counter electrode, and the reference electrode is independently formed of gold or platinum.

4. The clinical bedside system of claim 1, wherein the antifouling membrane over each working electrode of the working electrode(s) includes a conducting polymer independently selected from polyethylenimine; poly(3-methylthiophene); poly-5,2′-5′,2″-terthiophene-3′-carboxylic acid; polyaniline; polyaniline-poly(acrylic acid); polypyrrole-polyvinyl sulfonate; polyanion-doped poly(pyrrole); and poly(o-phenylenediamine).

5. The clinical bedside system of claim 1, wherein the capture antibody is either directly immobilized on the antifouling membrane or indirectly immobilized on the antifouling membrane through streptavidin or avidin.

6. The clinical bedside system of claim 1, wherein the capture antibody is a biotinylated antibody.

7. The clinical bedside system of claim 1, wherein the enzyme conjugated to the detection antibody is horseradish peroxidase (“HRP”), alkaline phosphatase (“ALP”), β-galactosidase, acetylcholinesterase (“AchE”), or catalase.

8. The clinical bedside system of claim 1, wherein each antibody of the capture antibody and the detection antibody is an anti-biomarker antibody for recognizing the biomarker and sandwiching the biomarker between the capture antibody and the detection antibody.

9. The clinical bedside system of claim 8, wherein the biomarker is a pro-inflammatory mediator selected from an interleukin (“IL”) including at least IL-1α, IL-1β, IL-6, IL-8, IL-11, IL-12, IL-17, IL-18, a member of the IL-20 family, or IL-33; tumor necrosis factor-α (“TNF-α”); leukemia inhibitory factor (“LIF”); interferon-γ (“IFN-γ”); oncostatin M (“OSM”); ciliary neurotrophic factor (CNTF); transforming growth factor-β (“TGF-β”); granulocyte macrophage colony-stimulating factor (“GM-CSF”); and other immunoregulatory biomolecules that attract inflammatory cells.

10. The clinical bedside system of claim 8, wherein the biomarker is an anti-inflammatory mediator selected from an interleukin including at least IL-1 receptor antagonist (“IL-1Ra”), IL-4, IL-6, IL-10, IL-11, or IL-13; and other immunoregulatory biomolecules that prevent potentially harmful effects of persistent or excess inflammatory reactions.

11. The clinical bedside system of claim 8, wherein the biomarker is procalcitonin (“PCT”), lactate, or C-reactive protein (“CRP”).

12. The clinical bedside system of claim 8, wherein the biomarker is angiopoietin-1 (“ANG-1”), angiopoietin-2 (“ANG-2”), plasminogen activator inhibitor-1 (“PAI-1”), tissue inhibitor of metalloproteinase-2 (“TIMP-2”), insulin-like growth factor-binding protein-7 (“IGFBP7”), soluble tumor necrosis factor receptor-1 (“sTNFR1”), receptor for advanced glycation endproducts (“RAGE”), decoy receptor 3 (“Dcr3”), soluble CD163, delta-like protein 1 (“DLL1”), hyaluronan, or syndecan.

13. The clinical bedside system of claim 1, wherein each working electrode of the working electrode(s) is configured to detect a unique biomarker, thereby providing a suite of biomarkers for both precision and accuracy.

14. The clinical bedside system of claim 1, wherein the medical device is a central venous catheter, an indwelling urinary catheter, or a skin-contacting wearable medical device.

15. A biosensor for complex care patients, comprising:
an inert substrate;
one or more working electrodes (“working electrode[s]”) deposited on the substrate, each working electrode of the working electrode(s) having an antifouling membrane thereover to which a capture antibody or a homogenous population of capture antibodies including the capture antibody is immobilized, the capture antibody configured to capture a biomarker between it and a detection antibody of a homogenous population of detection antibodies including the detection antibody, thereby sandwiching the biomarker between the capture antibody and the detection antibody for a detectable redox reaction between an enzyme conjugated to the detection antibody and its corresponding working electrode;
a counter electrode deposited on the substrate such that the counter electrode completes one or more electrical circuits (“electrical circuit[s]”) including the working electrode(s), respectively; and
an optional reference electrode deposited on the substrate such that the reference electrode is operably connected to the electrical circuit(s), the reference electrode configured to provide a reference point against which changes in potential at the working electrode(s) are measured.

16. The biosensor of claim 15, wherein each electrode of the working electrode(s), the counter electrode, and the reference electrode is independently formed of gold or platinum.

17. The biosensor of claim 16, wherein the antifouling membrane over each working electrode of the working electrode(s) is a conducting polymer independently selected from polyethylenimine; poly(3-methylthiophene); poly-5,2′-5′,2″-terthiophene-3′-carboxylic acid; polyaniline; polyaniline-poly(acrylic acid); polypyrrole-polyvinyl sulfonate; polyanion-doped poly(pyrrole); and poly(o-phenylenediamine).

18. The biosensor of claim 17, wherein the capture antibody is either directly immobilized on the antifouling membrane or indirectly immobilized on the antifouling membrane through streptavidin or avidin.

19. The biosensor of claim 15, wherein the capture antibody is a biotinylated antibody.

20. The biosensor of claim 15, wherein the enzyme conjugated to the detection antibody is horseradish peroxidase (“HRP”), alkaline phosphatase (“ALP”), β-galactosidase, acetylcholinesterase (“AchE”), or catalase.

21. The biosensor of claim 15, wherein each antibody of the capture antibody and the detection antibody is an anti-biomarker antibody for recognizing the biomarker and sandwiching the biomarker between the capture antibody and the detection antibody.

22. The biosensor of claim 21, wherein the biomarker is a pro-inflammatory mediator selected from an interleukin (“IL”) including at least IL-1α, IL-1β, IL-6, IL-8, IL-11, IL-12, IL-17, IL-18, a member of the IL-20 family, or IL-33; tumor necrosis factor-α (“TNF-α”); leukemia inhibitory factor (“LIF”); interferon-γ (“IFN-γ”); oncostatin M (“OSM”); ciliary neurotrophic factor (CNTF); transforming growth factor-β (“TGF-β”); granulocyte macrophage colony-stimulating factor (“GM-CSF”); and other immunoregulatory biomolecules that attract inflammatory cells.

23. The biosensor of claim 21, wherein the biomarker is an anti-inflammatory mediator selected from an interleukin including at least IL-1 receptor antagonist (“IL-1Ra”), IL-4, IL-6, IL-10, IL-11, or IL-13; and other immunoregulatory biomolecules that prevent potentially harmful effects of persistent or excess inflammatory reactions.

24. The biosensor of claim 21, wherein the biomarker is procalcitonin (“PCT”), lactate, or C-reactive protein (“CRP”).

25. The biosensor of claim 15, wherein each working electrode of the working electrode(s) is configured to detect a unique biomarker, thereby providing a suite of biomarkers for both precision and accuracy.
